(19) United States
(12) Patent Application Publication
(10) Pub. No.: US 2025/0176913 A1
(43) Pub. Date: Jun. 5, 2025

(54) RADIAL TISSUE STIFFNESS MEASUREMENT APPARATUS AND ANALYSIS TECHNIQUE

(71) Applicant: The Charles Stark Draper Laboratory, Inc., Cambridge, MA (US)

(72) Inventors: David W. Sutherland, II, Belmont, MA (US); Corin Williams, Framingham, MA (US); Aisling S. McEleney, Somerville, MA (US)

(73) Assignee: The Charles Stark Draper Laboratory, Inc., Cambridge, MA (US)

(21) Appl. No.: 18/841,195

(22) PCT Filed: Feb. 24, 2023
(86) PCT No.: PCT/US2023/013787

§ 371 Date: Aug. 23, 2024

Related U.S. Application Data
(60) Provisional application No. 63/314,122, filed on Feb. 25, 2022.

(51) Int. Cl. A61B 5/00

(52) U.S. Cl. CPC A61B 5/6853; A61B 5/0053

(57) ABSTRACT

A system can include a balloon catheter, a pump, a pressure sensor, and one or more processors, The balloon catheter can be configured to be inserted into a lumen of a tissue. The pump can be configured to provide fluid into the balloon catheter to cause the balloon catheter to induce a radial stress of the tissue. The pressure sensor can be configured to detect a pressure of the fluid while the balloon catheter is inducing the radial stress of the tissue. The one or more processors can be configured to determine a characteristic of the tissue based at least on the pressure.

### CROSS-REFERENCE TO RELATED APPLICATIONS

The present application claims the benefit of and priority to U.S. Provisional Application No. 63/314,122, filed Feb. 25, 2022, the disclosure of which is incorporated herein by reference in its entirety.

### GOVERNMENT SUPPORT STATEMENT

This invention was made with government support under the USAMRAA Technology/Therapeutic Development Award (W81XWH-20-1-0295) awarded by the Department of Defense. The government has certain rights in the invention.

### FIELD

Aspects and implementations disclosed herein are generally directed to systems and methods for stiffness measurements of tissues having lumens, for example, for the characterization of healthy tissue as well as detection and characterization of abnormalities.

### SUMMARY

At least one aspect relates to a method of tissue stiffness measurement in the radial direction. The method comprises inserting a balloon catheter into a lumen of the tissue, pressurizing the balloon catheter with a fluid to cause the balloon catheter to expand, press against of the tissue, and induce radial stress in the tissue, monitoring a change in pressure of the fluid while expanding the balloon catheter, and determining the stiffness of the tissue from the measurements of pressure and diameter changes of the tissue.

In some implementations, inserting the balloon catheter into the lumen of the tissue comprises inserting the balloon catheter into one of a blood vessel, a heart valve, or a portion of the digestive tract or respiratory tract.

In some implementations, the method is performed in a subject in vivo.

In some implementations, the method is performed without damaging the tissue.

In some implementations, pressurizing the balloon catheter with the fluid comprises pressurizing the balloon catheter with one of saline solution, water, air, or contrast agents.

In some implementations, the method further comprises imaging the balloon catheter and/or tissue during one or both of inserting the balloon catheter or pressurizing the balloon catheter.

In some implementations, the imaging is performed by one of echocardiogram, X-ray, digital photography, or MRI imaging. Imaging or measurements may be facilitated/aided by use of a contrast agent in the balloon or a radio opaque material used for the balloon or applied to the balloon.

In some implementations, the method further comprises assessing dimensions and strain of the tissue based on the imaging.

In some implementations, pressurizing the balloon catheter comprises pressurizing the balloon catheter to a pressure above which would be exerted on the radial tissue sample by in vivo physiological processes.

In some implementations, the method further comprises fabricating one of a growth-adaptable stent or a growth adaptable heart valve having a stiffness matching the stiffness of a region of interest of the tissue or selecting one of a growth-adaptable stent or a growth adaptable heart valve having a stiffness matching the stiffness of a region of interest of the tissue from a set of available options.

In some implementations, the method further comprises diagnosing the radial tissue for tissue fibrosis based on the stiffness of the radial tissue.

In some implementations, the method further comprises locating the tissue fibrosis with an imaging system or with stiffness measurements alone. Stiffness measurements indicative of the tissue fibrosis may correspond to known elevated results or localized stiffening relative to other points in the patient's body.

At least one aspect relates to a system for performing in vivo measurements of radial tissue stiffness. The system comprises a balloon catheter configured to be inserted into a lumen of the tissue of a subject, an infusion pump configured to deliver a fluid into the balloon catheter while the balloon catheter is disposed within the lumen of the tissue, a pressure sensor configured to monitor pressure of the fluid within the balloon catheter, an image capture system configured to monitor a position and change in shape of the balloon catheter and of the tissue during and subsequent to delivery of the fluid into the balloon catheter while the balloon catheter is disposed within the lumen of the tissue, and a controller configured to control the infusion pump, to analyze pressure data from the pressure sensor, and to determine the stiffness of the tissue from a change in pressure of the fluid while delivering the fluid into the balloon catheter.

In some implementations, the controller further includes software, which when executed by the controller, causes the controller to perform one or more of (i) tracking a volume of fluid infused into the balloon catheter, (ii) using measured volume and pressure in the balloon catheter to estimate diameter and strain of the tissue, or (iii) isolate tissue forces from transient or system pressure artifacts.

In some implementations, a balloon portion of the balloon catheter comprises a radio opaque material.

In some implementations, the controller further includes software, which when executed by the controller, causes the controller to perform any of the aspects or implementations of the method described above.

At least one aspect relates to a method. The method can include inserting a balloon catheter into a lumen of a tissue. The method can include providing a fluid into the balloon catheter to cause the balloon catheter to apply force against the tissue. The method can include monitoring a pressure of the fluid. The method can include determining a characteristic of the tissue based at least on the pressure and a size metric of the tissue.

In some implementations, the method includes increasing the pressure of the fluid by providing the fluid.

In some implementations, causing the balloon catheter to apply force against the tissue comprises inducing radial stress in the tissue.

In some implementations, the characteristic of the tissue includes at least one of a stiffness of the tissue or an indication of a pathological condition of the tissue.

In some implementations, the balloon catheter is inserted into the lumen of the tissue in vivo.

In some implementations, inserting the balloon catheter into the lumen of the tissue comprises inserting the balloon catheter into one of blood vessel, a heart valve, a vagina, a portion of a respiratory tract, or a portion of a digestive tract.

In some implementations, the method includes detecting an image of the balloon catheter and the tissue.

In some implementations, determining the size metric of the tissue based at least on the image.

In some implementations, monitoring the pressure of the fluid includes detecting the pressure using a pressure sensor coupled with the balloon catheter.

In some implementations, the method includes detecting a fibrosis condition of the tissue based at least on the characteristic of the tissue.

At least one aspect relates to a system. The system can include a balloon catheter configured to be inserted into a lumen of a tissue. The system can include a pump configured to provide fluid into the balloon catheter to cause the balloon catheter to induce a radial stress of the tissue. The system can include a pressure sensor configured to detect a pressure of the fluid while the balloon catheter is inducing the radial stress of the tissue. The system can include one or more processors configured to determine a characteristic of the tissue based at least on the pressure.

In some implementations, the pump can increase the pressure of the fluid by pumping the fluid into the balloon catheter.

In some implementations, the one or more processors can determine the characteristic of the tissue based at least on the pressure and a size metric the size metric corresponding to a diameter of at least one of the lumen or the balloon catheter.

In some implementations, the characteristic of the tissue includes at least one of a stiffness of the tissue or an indication of a pathological condition of the tissue.

In some implementations, the balloon catheter includes a radio opaque material.

In some implementations, the balloon catheter includes the pressure sensor.

In some implementations, the one or more processors can detect an indication of tissue fibrosis based at least on the characteristic of the tissue.

In some implementations, the one or more processors can determine the characteristic of the tissue based at least on an image of the balloon catheter and the tissue.

In some implementations, the one or more processors can determine the size metric of the tissue based at least on the image.

In some implementations, the image includes at least one of an echocardiogram, an X-ray image, a camera image, or an MRI image.

Those skilled in the art will appreciate that the summary is illustrative only and is not intended to be in any way limiting. Any of the features described herein may be used with any other features, and any subset of such features can be used in combination according to various embodiments. Other aspects, inventive features, and advantages of the devices and/or processes described herein, as defined solely by the claims, will become apparent in the detailed description set forth herein and taken in conjunction with the accompanying drawings.

### DESCRIPTION OF THE DRAWINGS

The accompanying drawings are not intended to be drawn to scale. For purposes of clarity, not every component may be labeled in the drawings.

In the drawings:

FIG. 1 depicts an example of a system for measuring tissue stiffness;

FIG. 2 depicts an example of a region of a heart that was tested for size and stiffness;

FIG. 3A depicts an example of the location of a balloon catheter inserted into an major artery of a heart for size and stiffness measurements;

FIG. 3B depicts an example of a balloon catheter disposed in a pulmonary valve annulus of a heart and directions of expansion measured during testing;

FIGS. 4A-4C depict examples of results of pig artery stress-strain curve measurements;

FIG. 4D depicts an example of a chart illustrating changes in inner diameter of a pulmonary valve annulus of a pig heart in horizontal and vertical directions during testing;

FIGS. 5A and 5B illustrate results of calculations of Young's modulus of pig arteries in accordance with an example disclosed herein;

FIGS. 6A-6C depict examples of plots of Young's modulus vs. stress for radial and uniaxial testing methods for different pig artery samples;

FIG. 7 depicts an example of a system for in vivo detection of characteristics of subjects; and

FIG. 8 depicts an example of a method of in vivo detection of characteristics of subjects.

### DETAILED DESCRIPTION

Useful implantable medical devices may be designed based at least on the mechanical properties of tissue at a target implantation site. Existing ex vivo methods of soft tissue mechanical property characterization require that the tissue be destroyed, and therefore cannot be used in living tissue in situ for patient-specific applications, such as for real-time, near real-time, and/or nondestructive applications. Existing in vivo methods of soft tissue mechanical property characterization cannot apply forces outside of physiological ranges and therefore cannot collect data on how the additional forces induced by an implantable device might affect the tissue. Therefore, there is a need for a method of characterizing the mechanical properties of soft tissue nondestructively and in excess of physiological ranges in the body.

There are many places in the body where devices such as stents may be implanted, including but not limited to various blood vessels (coronary, peripheral, etc.), the vagina, bile ducts, the urinary tract, and the digestive tract (including, e.g., esophagus, intestine). The tissue stiffness in these areas would be of interest to inform stent design, especially if the stent being designed is also growth-adaptable. Traditionally, it is not an issue if cardiovascular stents are much stiffer than the surrounding tissue because induced local fibrosis is balanced by the remaining elasticity in the rest of the cardiovascular circuit. A growth adaptable stent, however, should match tissue stiffness at the implantation site to prevent complications such as rapid expansion, fibrosis, or restenosis, deformation of the stent, or stent/device migration. Aspects and implementations disclosed herein may provide measurements of tissue stiffness at a location where a growth-adaptable stent may be implanted in a subject and allow for design of the growth-adaptable stent with a compliance matching that of the tissue in the area it is to be implanted.

Additionally, aspects and implementations disclosed herein can aid in the diagnosis of internal tissue fibrosis. Tissue fibrosis is the pathological stiffening of tissue and is not limited to the cardiovascular system. It can happen in the airways, or in the esophagus and intestines as a result of eosinophilic esophagitis, prolonged exposure to allergens/irritants/acid reflux, scleroderma, rheumatoid arthritis, Crohn's disease, ulcerative colitis, myelofibrosis, and systemic lupus erythematosus. Esophageal fibrosis is extremely detrimental in children and typically requires a biopsy to diagnose. Intestinal fibrosis currently lacks a reliable method of diagnosis. Fibrosis can also occur in the vagina post radiation therapy. There is currently no reliable way to diagnosis this fibrosis, but ultrasound is used most often.

Aspects and implementations disclosed herein can clinical balloon catheter technology to perform various measurement applications. Balloon catheters can be deployed into the cardiovascular system and expanded to a set maximum diameter by applying an unmodulated high pressure to expand the balloon for size measurement or to expand the tissue for therapeutic purposes. Aspects and implementations disclosed herein include sensing, actuation, and control equipment that can be combined to extract tissue property information in real time throughout the entire balloon expansion process. This process can be automated (e.g., with computer control of the pump and pressure/strain/imaging sensors) and may be implemented with a commercially available balloon catheter or optimized for the purpose with a customized balloon geometry. Aspects and implementations of the disclosed technique provides a value for tissue stiffness for a relatively large area of tissue.

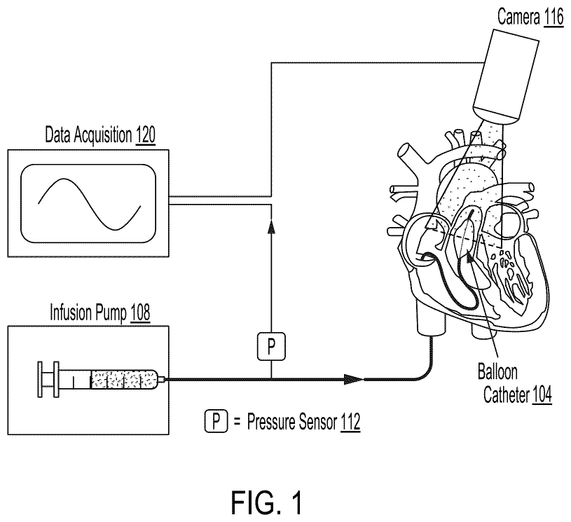

Radial stress testing utilized by aspects and implementations of the system and method disclosed herein allows for mechanical testing outside of physiological ranges with the potential to be used nondestructively in patients. Aspects and implementations can include a saline-filled balloon catheter 104, an infusion pump 108, a pressure sensor 112, an image capture system 116, and a controller/data acquisition unit 120 for controlling the infusion pump and reading pressure data from the pressure sensor and including analysis software for analyzing the data acquired during testing, as depicted in FIG. 1. The infusion pump is used to control the flow of saline in and out of the balloon catheter. The pressure sensor and data acquisition unit are used to track and record the changes in pressure and volume within the system. The balloon catheter is placed in the tissue orifice or lumen and inflated either steadily or in discrete increments to increase the pressure applied to the tissue. The response of the tissue to this pressure is imaged in vivo by, for example, an echocardiogram, X-ray, or MRI system. Imaging or measurements may be facilitated/aided by use of a contrast agent in the balloon or a radio opaque material used for the balloon or applied to the balloon. Images or videos may be taken and paired with the corresponding pressure condition.

In some implementations, image processing may be used to assess tissue dimensions and strain. Pressure induced by the balloon expansion may be characterized and subtracted from the in situ expansion values. Values from a control condition are subtracted from the measured response and the resulting pressure values can be converted to stress values using the hoop stress correlation equation (P=2St/D, where: P=pressure (psig); t=nominal wall thickness (inches); D=outside Diameter (inches); and S=allowable stress (psi)). The combination of multiple readings allows for assessment of the tissue stiffness over the applied force range.

Aspects and implementations of the disclosed system and method may be used nondestructively in vivo and can provide data on forces outside of physiological ranges, which can provide more accurate assessment of tissue stiffness, particularly those that are diseased (e.g., fibrosis, calcification). For tissues without a reliable physiologic stress cycle (e.g., a cardiac cycle) it may be the only way to acquire stiffness measurements. Additionally, the disclosed solution provides diagnostic capabilities for fibrotic diseases that currently lack reliable or minimally invasive approaches. Systems and methods in accordance with the present disclosure can be applied to detection of characteristics of tissues of various anatomical vessels and/or lumens of subjects, including but not limited to blood vessels (e.g., arteries, veins), heart valves, vaginal vessels, portions of respiratory tracts or other pulmonary vessels, or portions of digestive tracts or other gastrointestinal vessels; and can be implemented for detecting various conditions of tissue, such as fibroses, tumors, lesions, or other pathologies.

### I. Example: Characterization of Main Pulmonary Artery and Valve Tissue of Piglets Using Echocardiography, Uniaxial Tensile Testing, and Balloon Catheter Sensor System

### A. Introduction

While geometric measurement of cardiac structures in vivo may be relatively straightforward, techniques to measure mechanical properties in situ remain limited. The mechanical properties of individual patients can be estimated noninvasively by imaging vascular deflection across the cardiac cycle, but cardiovascular devices that mechanically interact with tissue, such as self-expanding stents, require knowledge of tissue stress response in excess of physiologic ranges. Well established ex vivo techniques exist, such as uniaxial and biaxial tensile testing, which can provide mechanical information within and beyond physiologic stress regimes. However, they require excision of the tissue from its native site, which is inherently destructive. It has not been demonstrated that these methods replicate the stress response of living tissue in situ. Currently, there are no reported techniques that are capable of nondestructively obtaining patient-specific mechanical properties of tissue when stress regimes above physiologic ranges are of importance.

An objective of the work described herein was to develop a previously unavailable dataset primarily capturing diameter and stiffness measurements of juvenile porcine main pulmonary artery and the tissue region containing the pulmonary valve annulus that will inform pediatric heart valve design requirements for preclinical animal studies. To achieve this goal, the main pulmonary artery (PA) and pulmonary valve annulus (PV) regions of Yorkshire (YO) and Yucatan (YU) mini-swine piglets (weight range 5.8-15.3 kg, 40-52 days old) were characterized. Tissue dimensions were measured by echocardiography and blood pressure was recorded prior to excising the hearts for ex vivo mechanical testing. Tests of the PA and PV region tissues were first performed with an implementation of the balloon catheter-based radial method disclosed above, followed by excising the tissue for uniaxial testing. It was found that juvenile porcine populations provided appropriate cardiovascular geometries and tissue mechanical properties for modeling pediatric pulmonary implants. Additionally, results indicated that balloon catheter-based radial methodologies in accordance with the present disclosure can provide accurate tissue stiffness measurements, showing promise for continued development towards patient-specific non-destructive in situ measurement.

### B. Materials and Methods:

### 1. Tissue Preparation for Radial Testing:

YU and YO piglet hearts were stored at −80° C. for 1-2 months before ex vivo mechanical testing was performed. Previous studies have shown that freezing porcine cardiac tissue at this temperature results in no statistically significant changes to mechanical properties for up to 1 year. On the day of testing, a single heart was removed from the freezer and fully submerged in a beaker of room temperature water until fully defrosted. The bottom third of the heart was removed with a transverse cut to provide simplified access to the PV region and PA through the right ventricle. The connective tissue between the PA and the surrounding large vessels was severed. The heart was then submerged in PBS to avoid dehydration during testing. The PA and PV region of the heart was tested on the same day using both radial and uniaxial testing techniques. This preparation method was repeated for each heart on its respective testing days.

### 2. Radial Testing System:

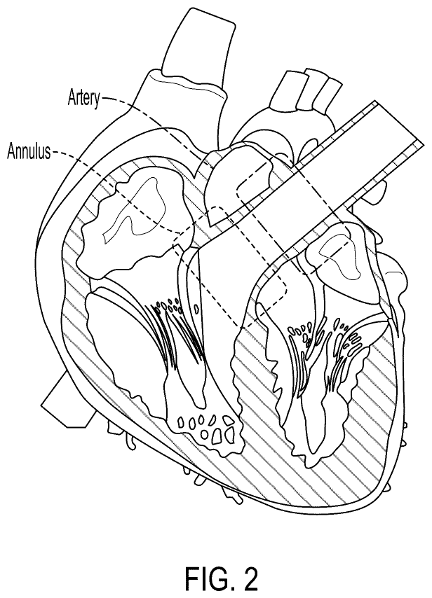

A TYSHAK II 22 mm balloon catheter (NuMed for Children, Orlando, FL) was connected to a 20 mL syringe (Plastipak, BD, Franklin Lakes, NJ) mounted in a syringe pump (PHD Ultra, Harvard Apparatus, Holliston, MA) with 1/16″ Tygon tubing (Saint Gobain, Courbevoie, France). A barbed tee-junction was used to connect a Luer pressure sensor (Pendo TECH, Princeton, NJ) between the syringe and catheter perpendicular to the flow. Pressure data was recorded using the AcqKnowledge software package and a MP150 data acquisition system (BIOPAC, Goleta, CA). The fluid circuit was purged of air with distilled water. Digital cameras (Dino-Lite Edge AM4115ZTF, Dino-Lite, Torrance, CA and SX40 HS, Canon, Tokyo, Japan) were mounted on perpendicular axis with respect to each other and the balloon catheter guide wire axis to record balloon expansion for PA measurements. A single digital camera was mounted axially with respect to the balloon catheter guide wire for the PV measurements. The regions of interest for testing are indicated in FIG. 2.

### 3. Radial Measurement and Testing Procedure:

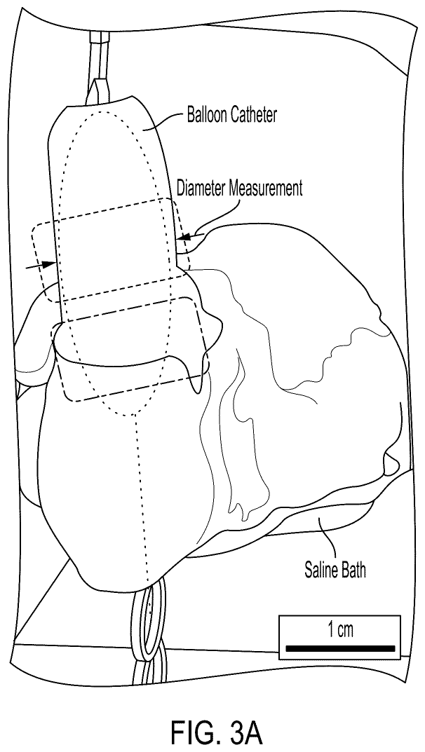

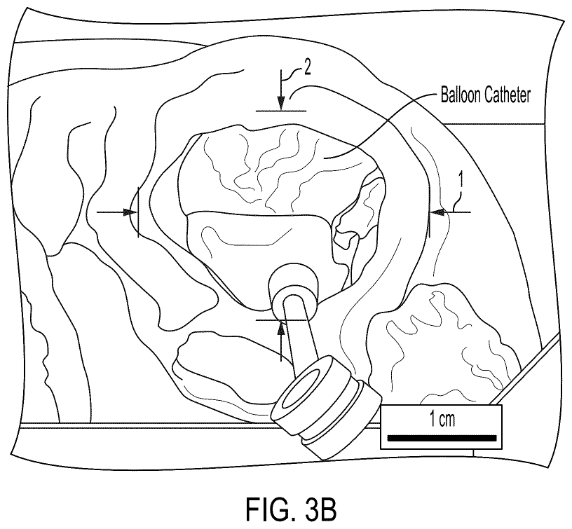

The balloon catheter was inserted into the main PA through the right ventricle such that the center of the balloon was aligned ˜1 cm above the PV. (See FIG. 3A.) The PA tissue was preconditioned five times before measurements were recorded to reduce tissue hysteresis. To precondition, the balloon was inflated at a flowrate of 1 mL/min until reaching 90 mmHg and the total infused volume was recorded. The same volume was then removed to return the tissue to an unstressed state. The recorded volume was infused and removed four additional times. The tissue measurement was performed by inflating the balloon within the PA a final time, at the same flowrate, to 90 mmHg. Time-stamped pressure data and video were recorded during the test. The balloon catheter was then removed from the heart, and the balloon was inflated without the tissue using the same procedure. Data collected in this step was later used as a baseline measurement in the radial data processing phase described below. The full set of preconditioning and test cycles occurred over the course of 40-50 minutes while the BIOPAC recorded pressure at a rate of 2 Hz.

After testing, the PA was excised from the heart with a transverse cut ˜0.5 cm above the PV leaflets. The balloon catheter was then placed inside the heart such that the leaflets were positioned at the center of the balloon. Preconditioning and measurement of the PV were performed as described above, and balloon expansion was recorded axially as described in the previous section to record PV diameter changes during testing. (See FIG. 3B.)

All YU hearts were measured using the optimized procedure described above. YO hearts, which were tested first, were preconditioned to 50 mmHg and inflated to 150 mmHg. Adjustments to the protocol were made to minimize damage that might occur during radial and uniaxial testing procedures.

### 4. Tissue Isolation and Measurements for Uniaxial Testing:

After radial testing, the tissue was prepared for uniaxial testing. The PA and PV annulus region were tested separately on the uniaxial setup. The PA was isolated from the heart as described in the previous section, and the branches were removed from the top. A vertical cut was made down the posterior side of the artery to unfurl the PA and lay it flat. The length of this cut was recorded as the length of the main PA. From this flat specimen, a rectangular strip with a width of 1 cm was cut in the circumferential direction. The length of this strip of tissue was recorded as the PA circumference. PA sample thickness and width measurements were taken at three equidistant points along the strip and averaged to account for variability in sample dimensions.

The PV region was isolated from the heart by making a transverse incision 1 cm below the previously described incision that separated the PA from the right ventricle. An incision straight through the aorta and right ventricle to the outside of the heart was performed to unfurl the PV region. The excess tissue surrounding the PV region was removed, except for on the ends (˜5 mm) of the tissue strip for clamping. Circumference (length), thickness, and width measurements were taken at three equidistant points along the specimen and averaged for each dimension. This step quantified the non-uniformity in specimen dimensions, specifically for the PV region which has variation in thickness along its length.

### 5. Uniaxial Tensile Testing System:

The tissue samples were mounted on the uniaxial testing system between two custom 3D printed tissue clamps. These clamps had surfaces textured in a checked pattern to minimize slippage and were tightened by hand. The static clamp was attached to a Mark-10 M3-2 Digital Force Gauge (Mark-10, Copiague, NY) and the dynamic clamp was affixed to the carriage of a linear stage actuator (5236A17, McMASTER-CARR, Douglasville, GA). This carriage was actuated towards and away from the force gauge using a NEMA 23 Unipolar Stepper Motor (1477, Pololu Corporation, Las Vegas, NV). The stepper motor's motion was dictated by an Arduino Uno Rev 3 (Arduino, Somerville, MA), which was programmed with the length of each specimen to calculate the preconditioning and testing sequence. The extension rate of the system (0.06 mm/s) was used to calculate the specimen's displacement.

### 6. Uniaxial Tensile Testing Procedure:

The tissue samples were mounted with approximately 5 mm of tissue held in each clamp and adjusted to remove slack without producing a force reading. The starting distance between the clamps was then measured and recorded. Three blue microbeads were then placed on the sample: one on each side where the tissue met the clamps to show if slippage occurred, and one marker bead in the center.

Preconditioning cycles were performed five times to 30% strain at a rate of 0.06 mm/s. Then the sample was stretched until fracture (test cycle) at the same extension rate. The full set of preconditioning and test cycles occurred over the course of approximately 30 minutes while the force gauge recorded tensile stress values at a rate of 1 Hz. The exposed surface of the tissue was hydrated manually for the duration of testing using a syringe filled with PBS.

All YU hearts were tested using the procedure described above. YO hearts were preconditioned ten times instead of five. Preconditioning cycles were reduced from ten to five between testing the YO and YU specimens to shorten the total testing time: data indicates that five cycles is sufficient. Additionally, YO extension rates were 0.6 mm/s rather than 0.06 mm/s for both preconditioning and test cycles. Adjustments to the testing protocol were made after YO data was obtained to better match radial and uniaxial testing conditions.

### 7. Radial Data Processing:

The baseline pressure dataset was subtracted from the corresponding tissue pressure dataset to isolate the pressure induced by the tissue's stress response. Images were taken from the digital recordings at 10 sec increments for the PA data sets and 5 sec increments for the PV data sets and aligned with the corresponding pressure measurements. The time increments were reduced for the PV to provide more data points since less volume was needed to reach 90 mmHg pressure. Vessel diameters were extracted from the images using ImageJ (NIH, Bethesda, MD). The external vessel diameter was identified for PA samples and the average PA thickness was used to estimate the internal diameter. The internal diameter was calculated directly for the PV samples. Diameter measurements were recorded in two perpendicular axes for all samples and averaged. Engineering strain was calculated at each time point using the vessel internal radius (Eq. 1). Engineering stress was calculated at each time point using the thin-walled hoop stress equation (Eq. 2).

$${ε}_{h} = \frac{{r}_{i} - {r}_{o}}{{r}_{o}} \qquad ( 1 )$$

$${σ}_{h} = \frac{p * {r}_{i}}{{t}_{i}} \qquad ( 2 )$$

Where εₕ is the engineering strain calculated using the hoop stress equation, rᵢ is the radius at each time point, and r₀ is the initial unstressed radius. σₕ is the engineering stress calculated using the hoop stress equation, p is the pressure at each time point, rᵢ is the radius at each time point, and tᵢ is the circumferential wall thickness.

### 8. Tensile Data Processing:

Force gauge readings, Fᵢ, were paired to their corresponding displacement values by time stamp. Time values were multiplied by the extension rate (0.06 mm/s) to determine the sample's displacement at the given point in time. The sample's cross-sectional area, A₀, was calculated by multiplying the initial average width measurement by the initial average thickness measurement. By dividing each force gauge reading by the initial cross-sectional area, axial engineering stress, σₐ, was calculated (Eq. 3). Similarly, each displacement value was divided by the sample's initial length, L₀, to calculate axial engineering strain, εₐ (Eq. 4).

$${σ}_{a} = \frac{{F}_{i}}{{A}_{0}} \qquad ( 3 )$$

$${ε}_{a} = \frac{Δ x}{{L}_{0}} \qquad ( 4 )$$

### 9. Calculation of Tissue Stiffness:

Tissue stiffness (Young's modulus) was estimated using a linear approximation of the elastic and fibrous regions of each sample's stress-strain curve. Linear fits and residuals were calculated in the NumPy library for Python (Wilmington, DE) using the polyfit method and correlation coefficient function respectively. Linear fits were calculated over a stress range of 10 kPa for both the elastic and fibrous regions. The stress ranges were selected to provide the highest quality approximation for each tissue type (PA and PV) and stress regime (elastic and fibrous). Fit quality was determined by residual squared values, R², which were averaged across the experimental group. The stress range associated with the highest average R² values was selected as the optimal linear approximations. Both YU and YO breeds were grouped together by tissue type (PA and PV region) to define elastic and fibrous regimes and compare Young's modulus measurements. For the PA tissue samples, 3-13 kPa and 26-36 kPa were selected for the elastic and fibrous regions, respectively. For the PV tissue samples, 0-5 kPa and 10-20 kPa were selected for the elastic and fibrous regions, respectively. Note that the PV elastic stress range is less than 10 kPa due to the relatively rapid onset of tissue transition when compared to the PA tissue.

Additionally, an average stress response curve was produced to compare the porcine data to additional data sets provided in other publications. Due to the large strain variations, a strain-averaged method was used to solve for the average tissue strain for the common applied stress range. The standard deviation of the strain was calculated and is presented as a horizontal error when plotted as a standard stress response curve.

### 10. Curve Fit Analysis:

A curve fitting analysis was also performed on the stress response measurements for each tissue sample to provide an instantaneous Young's modulus across the elastic, transitional, and fibrous regimes. A non-linear regression analysis was performed using the Curve Fitting Toolkit in MATLAB. The full data sets were used for the radial technique and the uniaxial techniques were truncated at a stress of 60 kPa to align with the available radial technique stress range. A two-term exponential method (Eq. 5) was chosen for all data sets and a residual analysis was performed to determine regression error:

$$ε = {ae}^{b σ} + {ce}^{d σ} \qquad ( 5 )$$

where a, b, c, and d are the output coefficients of the regression analysis, and ε is the instantaneous strain. Instantaneous Young's modulus values were calculated from the curve fit equations by taking the inverse of the first derivative.

### 11. Statistical Analysis:

A variety of statistical analyses were performed, as appropriate, using GraphPad Prism software (version 9.3.1). For comparisons of two groups, the Student's t-test was used. A two-way ordinary analysis of variance (ANOVA) with Tukey's post-hoc test was used to compare Young's modulus in the elastic and fibrous regimes as well as compare values measured within each regime by the radial and uniaxial testing methods. For the comparison of Young's modulus measured by the radial and uniaxial methods as a function of pressure, the multiple unpaired t-test with Welch correction and assumption of individual variance for each group was used. Differences were considered statistically significant for p-values<0.05. Bland-Altman analysis, specifically the difference vs. average method, was used to assess the limits of agreement between the radial and uniaxial testing methods for determining Young's modulus in the elastic and fibrous regimes of the three tissues (YU PA, YU PV, YO PA). Outliers in this analysis were noted but not removed.

### C. Results:

### 1. Tissue Mechanical Properties:

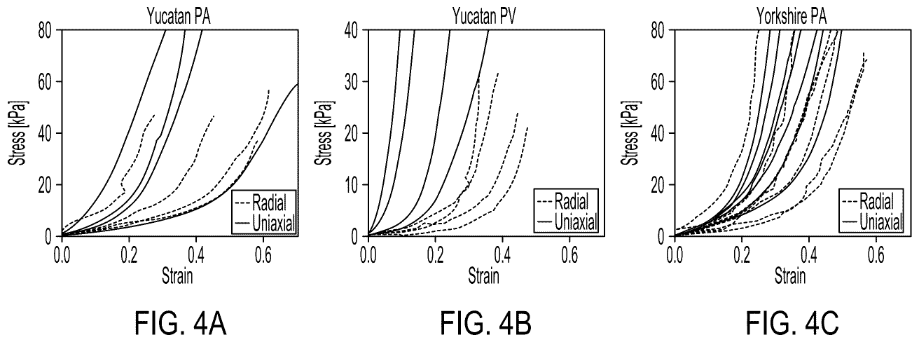

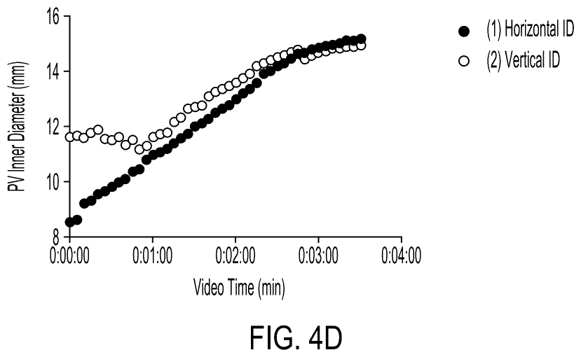

Experimental stress and strain data is shown for all breeds, tissues, and techniques in FIGS. 4A-4C. FIG. 4A illustrates the data for the Yucatan pulmonary artery (PA) tissue samples. FIG. 4B illustrates the data for the Yucatan pulmonary valve (PV) tissue samples. FIG. 4C illustrates the data for the Yorkshire PA tissue samples. It was observed that the radial method produced large strain values for the YU PV experimental group. Unlike the PA tissue, a non-negligible amount of pressure was required to lift the PV tissue and restore it to a cylindrical shape. This was observed in the recorded expansion data as the horizontal measurement axis reduced and the vertical axis rapidly increased until a discrete point in both curves where a constant relationship was reached and maintained for the remainder of the experiment. (See FIG. 4D.) This point was determined individually for each sample and the data was adjusted such that both stress and strain were reset to 0 at that point. The adjusted stress value for the YU PV data set was 1.38±0.40 kPa.

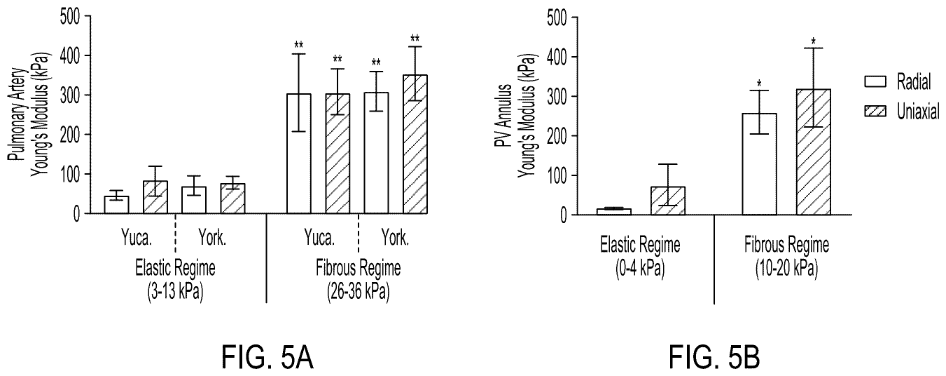

Young's modulus values were estimated by linear regression for the elastic and fibrous regimes of the stress-strain curves. FIG. 5A illustrates a comparison of Young's Modulus values determined by the radial and uniaxial testing methods for the pulmonary artery of both Yucatan and Yorkshire piglets. FIG. 5B illustrates a comparison of Young's Modulus values determined by the radial and uniaxial testing methods for the pulmonary artery of the pulmonary valve annulus of Yucatan piglets.

The elastic and fibrous regimes were estimated to be 3-13 kPa and 26-36 kPa respectively for PA tissue samples and 0-4 kPa and 10-20 kPa respectively for PV tissue samples. The elastic and fibrous regimes for a given tissue, breed, and measurement technique were significantly different (p<0.0005). There was no statistical difference between breed (YU vs. YO) or measurement technique (radial vs. uniaxial) within the respective elastic or fibrous regimes for the PA and PV data sets.

### 2. Instantaneous Tissue Stiffness:

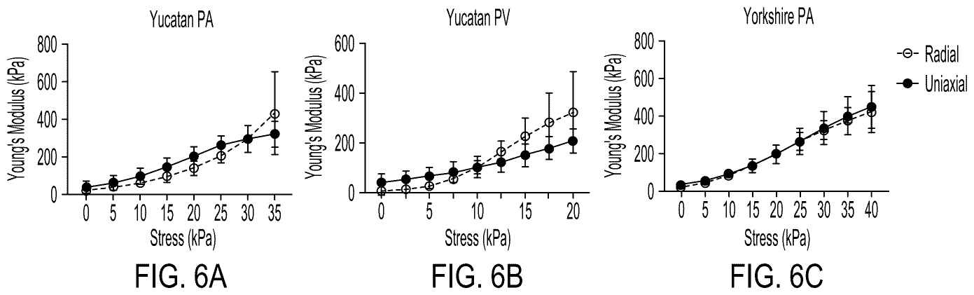

The exponential curve fit analysis allowed for the Young's modulus to be calculated at any point along the experimental stress response curve. In addition, it served to smooth the effects of experimental noise in the data sets. This approach may provide a more accurate assessment of the instantaneous Young's modulus compared to linear approximations. However, when comparing Young's modulus values between individual test curves within the same population, the large strain variation limited the ability to plot average Young's modulus against tissue strain. It was observed that plotting Young's modulus versus applied stress provided a much better alignment of techniques within our sample population. (See FIGS. 6A-6C. FIG. 6A is a plot of Young's modulus vs. stress for radial and uniaxial testing methods for Yucatan piglet pulmonary artery (PA). FIG. 6B is a plot of Young's modulus vs. stress for radial and uniaxial testing methods for Yucatan piglet pulmonary valve annulus (PV). FIG. 6C is a plot of Young's modulus vs. stress for radial and uniaxial testing methods for Yorkshire pulmonary artery (PA).) There was no significant difference between Young's modulus values determined by radial and uniaxial techniques at any given stress, further indicating good agreement between these methods.

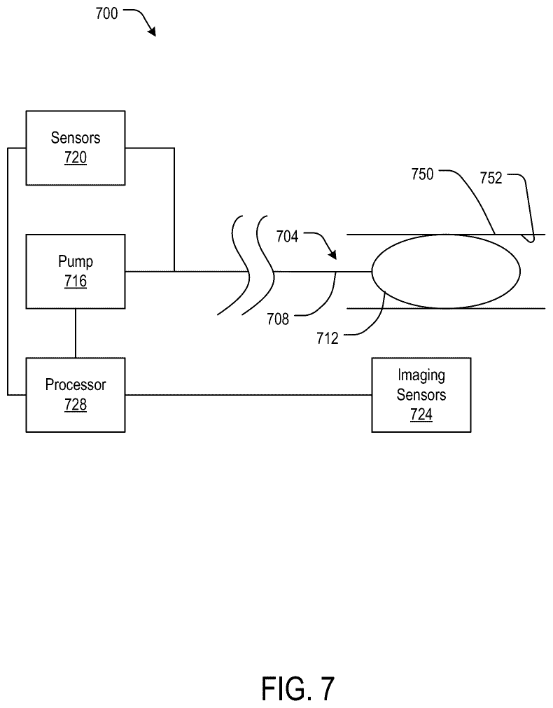

FIG. 7 depicts an example of a system 700. The system 700 can be used to detect mechanical properties or characteristics of tissues in subjects, such as stiffness or other characteristics of response to stress or strain (e.g., tensile strength, Young's modulus, yield strength, etc.). The system 700 can be used to detect various characteristics of tissues that may be related to mechanical properties of responses of tissues to stress or other forces, such as biological variability or age differences. The system 700 can be used to detect pathological conditions, such as fibrosis or cancer conditions, as well as to configure devices for deployment in the tissues, such as growth adaptive stents. The system 700 can detect or diagnose various such characteristics, properties, conditions, or pathologies based on one or more thresholds, models, rules, algorithms, policies, or functions, including but not limited to by using thresholds or calibration functions determined from experimental data or subject data indicative of various characteristics (e.g., based on thresholds or calibration functions that relate tissue stiffness with whether a condition is present, such as to classify tissue according to the tissue stiffness). The system 700 can incorporate features of various systems and devices described herein, such as the system or components thereof described with reference to FIG. 1. The system 700 can be used to facilitate an automated process for characterizing tissues of subjects in vivo, including to detect characteristics of the tissue that can be used to diagnose conditions of the subject and/or configure treatments for the subject.

The system 700 can include at least one catheter 704. The catheter 704 can be deployed to be inserted in a subject, such as to be positioned in a lumen 752 of a tissue 750 of the subject. The catheter 704 can be deployed in lumens 752 of any of various vessels of tissues 750 of the subject, including but not limited to a blood vessel, a heart valve, a vagina, a portion of a respiratory tract, or a portion of a digestive tract.

The catheter 704 can be a balloon catheter. For example, the catheter 704 can include a tube 708 coupled with a balloon member 712. The balloon member 712 can include a resilient material to enable the balloon member 712 to be expandable responsive to an increase of pressure in the balloon member 712. The tube 708 can define a passageway to allow for fluid delivery into the balloon member 712. In some implementations, the balloon member 712 is at least partially made of a radio opaque material and/or has a radio opaque pattern formed on a surface of the balloon member 712, which can facilitate imaging the balloon member 712.

The system 700 can include at least one pump 716 coupled with the catheter 704. For example, the pump 716 can be coupled with a proximal end of the tube 708 to pump fluid, such as saline fluid, into (or out of) the balloon member 712, such as to increase (or decrease) pressure of fluid in the balloon member 712. The pump 716 can be an infusion pump. The pump can be a syringe pump.

The system 700 can include at least one sensor 720. The sensors 720 can include a pressure sensor coupled with at least one of the pump 716 or the catheter 704 to detect a pressure of fluid in the catheter 704, such as to detect the pressure of fluid in the balloon member 712 of the catheter 704.

In some implementations, the sensors 720 include at least one sensor integrated with the catheter 704. For example, one or more sensors 720 can be piezoelectric sensors and/or strain gauges, and can be formed in or on a surface of the balloon member 712, to detect a metric of at least one of pressure, stress, or strain of the balloon member 712 (e.g., resulting from the pump 716 increasing or decreasing pressure of fluid in the balloon member 712). For example, one or more strain gauges 720 coupled with the balloon member 712 can enable the system 700 to detect a strain associated with expansion of the balloon member 712 responsive to increasing pressure of the fluid due to pumping of the fluid by the pump 716, and can enable the system 700 to determine a mechanical characteristic of the tissue 750, such as stiffness, based at least on the strain (e.g., by identifying an amount of deflection or diameter change or other size metric corresponding to the strain) and the pressure detected by the pressure sensor 720.

Referring further to FIG. 7, the system 700 can include or be coupled with one or more imaging sensors 724. The imaging sensors 724 can include, for example and without limitation, camera, X-ray, CT, MRI, or ultrasound imaging sensors 724. The imaging sensors 724 can be used to detect one or more images of the subject, including images representing the balloon member 712 and the lumen 752 of the tissue 750.

The system 700 can include one or more processors 728. The one or more processors 728 can be used to perform various processes described herein, including but not limited to detecting one or more characteristics of the tissue 750 according to sensor data received from at least one of the sensors 720 or the imaging sensors 724. For example, the processor 728 can receive a value of pressure of fluid in the balloon member 712 from one or more pressure sensors 720 (e.g., change in pressure), and determine the characteristic of the tissue 750 based at least on the value of pressure and a size metric indicative of a shape change of at least one of the balloon member 712 or the tissue 750 such as diameter, deformation, displacement, or other changes in size or shape of the tissue 750. For example, the processors 728 can apply the value of the pressure and the size metric as input to one or more models, functions, or algorithms to determine the characteristic of the tissue 750.

The one or more processors 728 can control operation of the pump 716 to cause the pump 716 to pump the fluid into the balloon member 712. For example, the one or more processors 728 can control operation of the pump 716 responsive to user input indicative of a target pressure or volume of fluid, or according to a predetermined target pressure or volume of fluid. The one or more processors 728 can receive sensor data indicating the pressure of the fluid from the pressure sensors 720.

The one or more processors 728 can determine the characteristic of the tissue 750, such as a stiffness, based on one or more functions or algorithms that determine stiffness according to force and deflection information. For example, the one or more processors 728 can determine, from the pressure of the fluid (e.g., from change in fluid) and the size metric, a force associated with pressure applied by the balloon member 712 against the tissue 752 (e.g., force corresponding to radial stress), and can determine the deflection (e.g., change in shape of at least one of the balloon member 712 or the lumen 752) according to the size metric (e.g., according to a cross-sectional area detected from the image data from the sensors 724). In some implementations, the one or more processors 728 apply one or more filters to at least one of the pressure data or the image data. For example, the pressure data and/or the image data can be relatively noise or complex. The one or more processors 728 can apply various filters, such as low-pass, high-pass, band-pass, or various combinations thereof; the filters may be calibrated according to experimental data used to detect stiffness from sensor data, which may indicate portions of the sensor data that can effectively provide signal data (rather than noise data) for determining stiffness.

In some implementations, the one or more processors 728 determine the size metric according to blood pressure data detected from image data received from the imaging sensors 724. For example, the image data can be received from an ultrasound sensor 724, which can indicate information such as blood flow or blood pressure (e.g., variation of blood pressure while the fluid is pumped in the balloon member 712 by the pump 716). The one or more processors 728 can determine the size metric, such as diameter or change in diameter of the lumen 752, based at least on the blood flow or blood pressure data (e.g., using one or more functions or algorithms that relate (change in) blood pressure with (change in) vessel diameter). In some implementations, the fluid in the catheter 704 has a density gradient, which can enable the ultrasound sensor 724 to detect contrast data corresponding to the density gradient of the fluid.

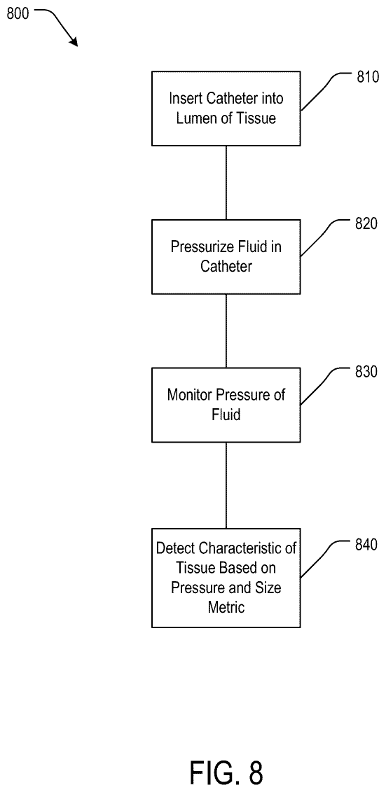

FIG. 8 depicts an example of a method 800 for in vivo detection of characteristics of tissues of subjects, such as for detecting mechanical characteristics such as stiffness. The method 800 can be performed using various systems and devices described herein, including but not limited to the systems described with reference to FIG. 1 and the system 700 described with reference to FIG. 7. The method 800 can be performed responsive to user inputs from various operators, including but not limited to medical professionals providing inputs to manual and/or automated controllers of one or more of a catheter delivery system (e.g., a guide-wire based system), an infusion pump coupled with a balloon member of a catheter coupled with the catheter delivery system, and one or more imaging devices (e.g., MRI, ultrasound, camera, X-ray, CT, etc.).

At 810, a balloon catheter can be inserted into a lumen of a tissue. The balloon catheter can be coupled with a tube, and inserted using a catheter delivery system, such as a guidewire system. The lumen of the tissue can be a lumen or vessel of any of a variety of tissues of a subject, including a blood vessel, a heart valve, a vagina, a portion of a respiratory tract, or a portion of a digestive tract, such that the balloon catheter can be delivered in vivo. The balloon catheter can be delivered to a target site of a subject for characterization of the tissue at the target site, such as a site at which a condition of the tissue or the subject is to be evaluated, or a site at which a stent is to be provided, such as a growth adaptable stent.

At 820, fluid can be provided into the balloon catheter to cause the balloon catheter to apply force against the tissue, such as to induce radial stress in the lumen. For example, the fluid can be provided by a pump, such as an infusion pump, that is fluidly coupled with the balloon catheter. The fluid can be provided responsive to an input to send a control signal (e.g., from a user interface of a controller) to the pump. The control signal can indicate one or more of a duration of time to pump the fluid, a volume of fluid to pump, a target pressure to pump the fluid to, or various combinations thereof (e.g., the control signal can indicate a schedule of pressure over time for the fluid). The fluid can be provided to cause an expected amount of radial stress in the tissue. The pump can be iteratively controlled to pump the fluid, such as responsive to receiving a plurality of control signals, such as to enable an operator to monitor the pressure of the fluid or, using one or more imaging devices, a size metric of the balloon catheter or the lumen, and provide inputs for controlling the pump according to the pressure or the size metric.

At 830, the pressure of the fluid can be monitored using a pressure sensor. The pressure sensor can be fluidly coupled with at least one of the pump or the balloon catheter, such as to be coupled with a flow path between the pump and the balloon catheter (e.g., as depicted in FIG. 1). The pressure can be communicated from the pressure sensor to one or more processors of the controller or a device remote from the controller. The pressure sensor can periodically sample the pressure and output an indication of the pressure. In some implementations, the controller controls the pump according to the pressure.

At 840, a characteristic of the tissue can be determined based at least on the pressure and a size metric of the tissue. The characteristic can be determined as a stiffness or other mechanical characteristic of the tissue. The size metric can indicate one or more of deflection, shape changes, diameter, or cross-sectional area of at least one of the balloon catheter or the lumen of the tissue. The size metric can be determined based at least on image data (e.g., MRI, CT, X-ray, ultrasound, camera imaging) of the balloon catheter or volume data of the balloon catheter. The image data can be detected while the pump is operating and/or while the pressure in the balloon catheter is changing, which can facilitate detecting a mechanical response of the tissue to the pressure in order to determine the characteristic of the tissue. In some implementations, one or more filters are applied to pressure data and/or image data to facilitate real-time determination of the characteristic of the tissue.

In some implementations, the characteristic of the tissue can be used to diagnose one or more conditions, such as pathologies, of the tissue. For example, conditions such as fibroses, tumors or other indications of cancers or cancerous tissues, or lesions can be diagnosed. For example, the one or more processors can apply the characteristic of the tissue (e.g., stiffness, Young's modulus) as input to one or more functions, algorithms, heuristics, classifiers, or policies for diagnosing conditions to determine the condition. For example, the one or more processors can compare the determined characteristic to one or more thresholds indicative of whether a condition is present to diagnose the condition. In some implementations, the characteristic can be detected at a plurality of points in time to facilitate longitudinal evaluation of the characteristic and the tissue. For example, a first process (e.g., of deploying the balloon catheter at a site in a subject, pressurizing fluid in the balloon catheter to apply a force against the tissue at the site, and monitoring pressure of the fluid and a size metric of the balloon catheter and/or the tissue) can be performed to determine the characteristic at a first time, and a second process can be performed subsequent to the first process to determine the characteristic at a second time.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations.

While operations are depicted in the drawings in a particular order, such operations are not required to be performed in the particular order shown or in sequential order, and all illustrated operations are not required to be performed. Actions described herein can be performed in a different order.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device, etc.) may include one or more devices (e.g., RAM, ROM, Flash memory, hard disk storage, etc.) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit and/or the processor) the one or more processes described herein.

References herein to the positions of elements (e.g., “top,” “bottom,” “above,” “below”) are merely used to describe the orientation of various elements in the FIGURES. The orientation of various elements may differ according to other exemplary implementations, and that such variations are intended to be encompassed by the present disclosure. References herein to the order of elements (e.g., “first,” “second,” “third,” “fourth,” “fifth,” “sixth,” “seventh”) are merely used for ease of description relative to each element in the FIGURES

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation or implementation, and references to “an implementation,” “some implementations,” “one implementation” or the like are not necessarily mutually exclusive and are intended to indicate that a particular feature, structure, or characteristic described in connection with the implementation may be included in at least one implementation or implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

Systems and methods described herein may be embodied in other specific forms without departing from the characteristics thereof. For example, the removable sprinkler protection device 100 may be used in various sprinkler applications. Further relative parallel, perpendicular, vertical or other positioning or orientation descriptions include variations within +/−10% or +/−10 degrees of pure vertical, parallel or perpendicular positioning. References to “approximately,” “about” “substantially” or other terms of degree include variations of +/−10% from the given measurement, unit, or range unless explicitly indicated otherwise. Coupled elements can be electrically, mechanically, or physically coupled with one another directly or with intervening elements. Scope of the systems and methods described herein is thus indicated by the appended claims, rather than the foregoing description, and changes that come within the meaning and range of equivalency of the claims are embraced therein.

1. A method of tissue characteristic measurement, comprising:
inserting a balloon catheter into a lumen of a tissue;
providing a fluid into the balloon catheter to cause the balloon catheter to apply force against the tissue;
monitoring a pressure of the fluid; and
determining a characteristic of the tissue based at least on the pressure and a size metric of the tissue.

2. The method of claim 1, wherein causing the balloon catheter to apply force against the tissue comprises inducing, in the tissue, at least one of radial stress, longitudinal stress, compressive stress, or shearing.

3. The method of claim 1, wherein the characteristic of the tissue comprises at least one of a stiffness of the tissue or an indication of a condition of the tissue.

4. (canceled)

5. The method of claim 1, wherein inserting the balloon catheter into the lumen of the tissue comprises inserting the balloon catheter, in vivo, into one of a blood vessel, a heart valve, a vagina, a portion of a respiratory tract, or a portion of a digestive tract.

6. (canceled)

7. The method of claim 1, further comprising determining the size metric of the tissue based at least on an image of the balloon catheter and the tissue, the size metric indicative of at least one of a diameter, an area, a deformation, or a shape change of the tissue.

8. The method of claim 6, further comprising applying one or more filters to data indicative of the pressure and image data of the image prior to determining the characteristic.

9. (canceled)

10. The method of claim 1, further comprising detecting a fibrosis condition of the tissue based at least on the characteristic of the tissue.

11. A system, comprising:
a balloon catheter configured to be inserted into a lumen of a tissue;
a pump configured to provide fluid into the balloon catheter to cause the balloon catheter to induce a stress of the tissue;
a pressure sensor configured to detect a pressure of the fluid while the balloon catheter is inducing the stress of the tissue; and
one or more processors configured to determine a characteristic of the tissue based at least on the pressure.

12. The system of claim 11, wherein the pump is configured to increase the pressure of the fluid by pumping the fluid into the balloon catheter.

13. The system of claim 11, wherein the one or more processors are configured to determine the characteristic of the tissue based at least on the pressure and a size metric of at least one of the tissue or the balloon catheter, the size metric indicative of at least one of a diameter, an area, a deformation, or a shape change of the at least one of the tissue or the balloon catheter.

14. The system of claim 11, wherein the characteristic of the tissue comprises at least one of a stiffness of the tissue or an indication of a pathological condition of the tissue.

15. The system of claim 11, wherein the balloon catheter comprises at least one of a radio opaque material or a radio opaque pattern.

16. The system of claim 11, wherein the balloon catheter comprises the pressure sensor.

17. (canceled)

18. The system of claim 11, wherein the one or more processors are configured to determine the characteristic of the tissue based at least on an image of the balloon catheter and the tissue.

19. The system of claim 18, wherein the one or more processors are configured to determine a size metric of the tissue based at least on the image, wherein the image comprises at least one of an echocardiogram, an X-ray image, a camera image, or an MRI image.

20. The system of claim 11, wherein the one or more processors are configured to detect an indication of cancerous tissue based at least on the characteristic of the tissue.

21. A method of tissue stiffness measurement in the radial direction, comprising:
inserting a balloon catheter into a lumen of the tissue;
pressurizing the balloon catheter with a fluid to cause the balloon catheter to expand, press against of the tissue, and induce radial stress in the tissue;
monitoring a change in pressure of the fluid while expanding the balloon catheter; and
determining the stiffness of the tissue from the measurements of pressure and diameter changes of the tissue.

22-28. (canceled)

29. The method of claim 21, wherein pressurizing the balloon catheter comprises pressurizing the balloon catheter to a pressure above which would be exerted on the radial tissue sample by in vivo physiological processes.

30. The method of claim 21, further comprising diagnosing the radial tissue for tissue fibrosis based at least on the stiffness of the tissue.

31. (canceled)

32. The method of claim 30, further comprising locating the tissue fibrosis with tissue stiffness measurements alone.

33-35. (canceled)
